(19) United States
(12) Patent Application Publication
(10) Pub. No.: US 2021/0353693 A1
(43) Pub. Date: Nov. 18, 2021

(54) Live Biotherapeutics to Treat and Prevent Lung Conditions

(71) Applicant: SciBac Inc., Burlingame, CA (US)

(72) Inventors: Anthony F. Cann, Burlingame, CA (US); Jeanette M. Mucha, Burlingame, CA (US); Stacy M. Townsend, Burlingame, CA (US); Derik A. Twomey, Burlingame, CA (US); Gregory Govoni, Burlingame, CA (US)

(21) Appl. No.: 17/286,453

(22) PCT Filed: Oct. 16, 2019
(86) PCT No.: PCT/US2019/056617

§ 371 Date: Apr. 17, 2021

Related U.S. Application Data
(60) Provisional application No. 62/746,742, filed on Oct. 17, 2018.

(51) Int. Cl. A61K 35/747; A61K 35/745; A61K 35/744; A61K 35/742; A61K 35/741; A61P 11/00; A61P 31/04

(52) U.S. Cl. CPC A61K 35/747; A61K 35/745; A61K 35/744; A61P 31/04; A61K 35/741; A61P 11/00; A61K 35/742

(57) ABSTRACT

Methods and compositions are described for treatment of lung conditions using live biotherapeutics, in particular modified microbes, such as chimeric microbial hybrids or microbial mutants produced by environmental selective pressure and screened for characteristics that are therapeutically beneficial for treatment or prevention of lung conditions.

### CROSS-REFERENCE TO RELATED APPLICATIONS

This application claims the benefit of U.S. Provisional Application No. 62/746,742, filed on Oct. 17, 2018, which is incorporated by reference herein in its entirety.

### FIELD OF THE INVENTION

The invention relates to live biotherapeutic strains designed to treat and/or prevent lung conditions, such as chronic infections in cystic fibrosis patients. In particular, the live biotherapeutic includes one or more modified microbe(s), such as chimeric microbial hybrids or microbial mutants that are produced using selective pressure from environmental stressor(s).

### BACKGROUND

Lung health and disease are influenced by the lung microbiota. The diversity of lung conditions ranges from inflammatory disease, such as asthma or chronic obstructive pulmonary disease (COPD), to infectious diseases (like pneumonia), to lung cancer—with many of these diseases having life threatening consequences. For some of these diseases, a direct connection between a bacterial causal agent and the disease state is known. For others, bacterial agents are suspected but remain to be proven as causal. Until recently, the only way to treat bacterial-induced diseases was with antibiotics. While antibiotics have saved many lives and remain useful, their indiscriminate modes of action tend to eliminate beneficial and commensal bacteria along with the harmful strains. The consequences of this unintended, collateral damage to the diversity of the lung microbiota is now becoming better understood, as such diseases as cystic fibrosis (CF) show chronic infections and antibiotic use is linked to reduced lung microbiota diversity and poor clinical outcomes.

Cystic fibrosis is a recessive, inherited genetic disease present in one out of 2000 Caucasian births. Cystic fibrosis patients have an altered lung environment from one or more genetic mutation(s) in the CF transmembrane conductance regulator (CFTR) gene located on chromosome 7. A CFTR mutation causes the chloride channels in the affected person's cell membranes to malfunction. The decrease in chloride ions on the epithelial surface, in comparison to normal genotype lungs, results in thicker mucus and inefficiencies due to epithelial absorption of water by osmosis (Clunes, M., et al (2007) Drug Discov Today Dis Mech 4(2):63-72). This thicker mucus allows organisms such as Pseudomonas aeruginosa to form biofilms in CF lungs. In addition, CF patients' innate immune systems do not work to clear microorganisms effectively due to the altered environment. This leads to a higher content of DNA in CF lungs, which also contributes to thicker mucus (Wang, Y., et al (2014) Int J Biochem Cell Biol 52:47-57). Since pathogens can hide in the thicker mucus, or sputum, of CF patients, they can evade both antibiotics and the immune response. The highest prevalence of Staphylococcus aureus is found in children with CF between the ages of 6 and 10 years of age(LiPuma, J. (April 2010) Clinical Microbiology Reviews 23(2):299-323). As patients age, the incidence of P. aeruginosa increases from 30% of CF patients at the age of 10 years to 80% of patients having chronic P. aeruginosa infections by the age of 18. Although standard antibiotic treatment helps to control aggressive outbreaks, there is no treatment to completely remove the infections. Ninety percent of CF patients die from pulmonary disease, mainly because of chronic Pseudomonas infection (Gaspar, M. C., et al (2013) Eur J Clin Microbiol Infect Dis DOI 10.1007/s10096-103-1876-y).

There is a need for an alternative therapeutic to clear chronic lung infections, especially those manifested in lungs of CF patients. Further, the contribution of indiscriminate antibiotic use has far-reaching consequences and increases the spread of antibiotic resistance on a global scale. Safe and effective alternatives to antibiotics are desperately needed.

### BRIEF SUMMARY OF THE INVENTION

Methods, compositions, and kits are provided for treatment or prevention of lung (e.g., pulmonary and/or respiratory) conditions.

In one aspect, methods are provided for treatment or prevention of a lung condition. The methods include administering a therapeutically or prophylactically effective amount of a modified microbe (a live biotherapeutic), e.g., a chimeric microbial hybrid or a microbial mutant, produced by selective pressure, to an individual in need thereof, wherein the administration results in prevention, amelioration, or elimination of at least one symptom of a lung condition in the individual. For example, the modified microbe may possess characteristics such as, but not limited to, ability to: degrade a biofilm, such as a Pseudomonas biofilm; degrade mucin; competitively inhibit Staphylococcus aureus; and/or secrete a bacteriocin directed against Pseudomonas aeruginosa.

In one embodiment, the lung conditions includes a microbial infection. For example, the microbial infection may include a bacterial infection, such as an infection including one or more bacterial species from genera Pseudomonas, Staphylococcus, Burkholderia, Mycobacterium, Stenotrophomonas, Achromobacter, Ralstonia, Pandoraea, Escherichia, Mycobacterium, Moraxell, Staphylococcus, Enterococcus, Streptococcus, Veillonella, Prevotella, Propionibacterium, Haemophilus, and/or Listeria.

In one embodiment, the microbial infection is a Pseudomonas aeruginosa infection. In an embodiment, the P. aeruginosa infection is chronic P. aeruginosa infection.

In one embodiment, the microbial infection is a Staphylococcus aureus infection. In an embodiment, the S. aureus infection is chronic S. aureus infection.

In one embodiment, the lung condition includes a fungal infection, such as an infection including one or more fungal species from the genera Candida, Malassezia, Neosartorya, Saccharomyces, and/or Aspergillus.

In one embodiment, the lung condition includes a eukaryotic infection, such as one or more eukaryotic species from the genera Ascaris, Schistosoma, Toxoplasma, Cryptosporidium, Cyclospora, and/or Paragonimus.

In one embodiment, the lung condition includes a viral infection, such as one or more virus selected from Influenza, Respiratory syncytial virus (RSV), Coronavirus, Rhinovirus, a Parainfluenza virus, Adenovirus, Astrovirus, Calicivirus, and/or Parvovirus.

In one embodiment, the lung condition includes fibrotic disease (FD), such as cystic fibrosis (CF), idiopathic pulmonary fibrosis (IPF), or interstitial pneumonia.

In one embodiment, the lung condition includes an inflammatory disease, such as asthma, COPD, bronchiectasis, or pneumonia.

In one embodiment, the lung condition incudes an autoimmune disease, such as rheumatoid arthritis, lupus, sarcoidosis, scleroderma or Sjogren's syndrome (risk factors for pulmonary fibrosis (PF)).

In one embodiment, the lung condition includes cancer, for example, adenocarcinoma, squamous cell carcinoma, or large cell carcinoma.

In some embodiments, one or more modified microbe as described herein is administered in combination with one or more additional treatment to treat, prevent, or ameliorate at least one symptom of the lung condition. For example, the additional treatment may include an antibiotic, a bacteriophage, an antibody, a peptide, an enzyme, a sugar, a glycopolymer, bacteriocin, and/or spores.

In some embodiments, a therapeutically or prophylactically effective amount of a modified microbe as described herein is formulated in a solid dosage form, such as a dry powder, a liquid dosage form, such as a solution, or a semi-solid dosage form. In one embodiment, a solid or liquid dosage form is administered by inhalation. For example, a solid dosage form may be formulated for nebulization in the lung, e.g., for inhalation and release in the large airways, small airways, and/or respiratory bronchioles. In another embodiment, the modified microbe is administered intranasally, for example, as a nasal spray or lung/nasal irrigation solution.

In another aspect, pharmaceutical compositions are provided. The pharmaceutical compositions include a therapeutically or prophylactically effective amount of a modified microbe as described herein, and a pharmaceutically acceptable carrier, formulated for treatment or prevention of a lung condition.

In another aspect, dosage forms are provided that include pharmaceutical compositions as described herein. For example, the dosage forms may include a therapeutically or prophylactically effective dose or a percentage of a therapeutically or prophylactically effective dose of a pharmaceutical composition as described herein. The dosage forms may be solid, liquid, or semi-solid, for example, formulated as an inhaled solution or dry powder, a nasal spray, or lung/nasal irrigation solution.

In another aspect, kits are provided that include a pharmaceutical composition, e.g., a unit dose, and optionally, instructions for use or patient instructions regarding a method of treatment or prevention of a lung condition as described herein.

### BRIEF DESCRIPTION OF THE DRAWINGS

FIG. 1 shows F2BH2 (top 2 patches) and F2BH3 (bottom 2 patches) as white patches with clearance from anaerobic bacteriocin activity at 48 hours against a luciferase producing strain of Psuedomonas aeruginosa (Xen-5) spread on the plate; pictures from bioluminescence in an IVIS machine.

FIG. 2 shows alginate lyase activity of parental and modified strains depicted by visible zones of clearing on alginate agar plates.

FIG. 3 shows mucinase activity of parental and modified strains depicted by visible zones of clearing on mucin agar plates.

FIGS. 4A-4B shows carbohydrate metabolism profiles of parental and modified strains characterized by API 50 CH carbohydrate fermentation strips (bioMérieux, Inc., Marcy l'Etoile, France). FIG. 4A shows exemplary bacterial with sensitivity to live biotherapeutic LH1 bacteriocins demonstrated with a quantitative cross-streak assay. FIG. 4B shows exemplary bacteria sensitive to live biotherapeutic LH1 parental strain bacteriocins demonstrated with a quantitative cross-streak assay.

FIG. 5 shows modified cross-streak assay. After 48 hours of anaerobic growth, seven multidrug-resistant S. aureus strains were streaked across LH1 and the measurement of inhibited growth was recorded among others tested in Table I.

FIG. 6 shows two examples of the ETEST® indicating the level of antibiotic sensitivity of LH1 to cystic fibrosis associated antibiotics tobramycin (TB) and aztreonam (AZ) following 24-hours anaerobic incubation.

FIG. 7 shows qualitative antibacterial activity against S. aureus CF clinical isolate by modified strain LH1 and Lactobacillus parent strain in a qualitative drop test showing visible zones of inhibition on agar plates.

FIG. 8 shows a drop test with ethyl acetate whole cell extracts of WT, LH1, and vehicle control against P. aeruginosa CF clinical isolate.

FIG. 9 shows a drop test with ethyl acetate whole cell extracts of WT strain A, WT strain B, LH1, vehicle control against S. aureus CF clinical isolate.

FIG. 10 shows a drop test with crude ethyl acetate extracts (supernatant, pellet, and 3 kD filtered supernatant) against S. aureus CF clinical isolate.

FIG. 11 shows LH1 at WT parental strain kills and continues to inhibit growth of planktonic P. aeruginosa strain PA01 up to 8 hours when combined at a 2:1 ratio.

FIG. 12 shows the ability of LH1 to remove carbapenem-resistant P. aeruginosa (AR0243, CDC AR Bank) 3-day anaerobic biofilms. P. aeruginosa biofilms were rinsed and strained with crystal violet after treatment with 10⁵-10⁷ CFU/mL LH1. Residual biofilm was quantified via optical density or crystal violet stain solubilized in ethanol (OD595).

FIG. 13 shows the ability of LH1 compared to WT parent to remove carbapenem-resistant P. aeruginosa (AR0243, CDC AR Bank) 3-day anaerobic biofilms. P. aeruginosa biofilms were rinsed and strained with crystal violet after treatment with 10⁵-10⁷ CFU/mL LH1. Residual biofilm was quantified via optical density or crystal violet stain solubilized in ethanol (OD595).

FIG. 14 shows the ability of LH1 to grow in CF mucin media up to 4 hours. LH1 was inoculated into CF mucin media and grown for 4 hours anaerobically at 37° C. LH1 was quantified by plating for viable counts anaerobically.

FIG. 15 shows the ability of LH1 to grow in CF sputum up to 4 hours. LH1 was inoculated into CF sputum and grown for 4 hours anaerobically at 37° C. LH1 was quantified by plating for viable counts anaerobically.

FIG. 16 shows the ability of LH1 to reduce endogenous S. aureus in pooled CF sputum in a time-kill assay. Pooled CF sputum was treated with 10⁶-10⁸ CFU/mL LH1 and incubated anaerobically for 24 hours then plated for viable counts on S. aureus selective media (Vogel-Johnson Agar) showing dose and time dependent reduction of endogenous S. aureus.

FIG. 17 shows the ability of LH1 crude extract to reduce endogenous S. aureus in pooled CF sputum. Pooled CF sputum was treated 1:1 LH1 and incubated anaerobically for 24 hours then plated for viable counts on S. aureus selective media (Vogel-Johnson Agar) showing eradication of endogenous S. aureus following 1-hour of treatment.

FIG. 18 shows the ability of LH1 crude extract to reduce carbapenem-resistant P. aeruginosa in a growth inhibition assay. A lawn of P. aeruginosa treated with undiluted, 1:2 and 1:100 diluted LH1 crude extract and incubated aerobically for 24 hours showed inhibition of growth in a dose dependent manner compared to vehicle control.

FIG. 19 shows the physiochemical properties of the predicted protein resulting from the frameshift mutation in the permease gene. Properties analyzed include molecular weight, extinction coefficient, iso-electric point, net charge at physiological pH, estimated solubility and hydropathy (Hopp-Woods scale) along the amino acid sequence of the predicted protein.

FIG. 20 shows the hydrophobicity of the new predicted protein along its amino acid sequence using the Kyte-Doolittle scale. Alternating regions of hydrophobicity and hydrophilicity can be seen.

FIG. 21 shows the amplified PCR products used to sequence the region in the permease gene encoding the frameshift mutation. Primers Perm03-F (5′-GCCGCCATAAAGCAAATGATCA-3′) (SEQ ID NO:1) and Perm01-R (5′-AGCCATCATGAACCGTCTCTTC-3′) (SEQ ID NO:2) with the Hot Start Taq DNA polymerase (NEBiolabs) were used to amplify the PCR product along. Visualization of the PCR product was accomplished by staining the agarose gel electrophoresis of the PCR reactions with SYBR Safe DNA gel stain (Invitrogen). PCR products from the 10 colonies were purified and sent for Sanger sequencing.

FIG. 22 shows the pourability of cystic fibrosis patient sputum samples following 4-hour treatment with 1:10 dilution of 100× LH1 crude extract compared to vehicle control.

FIG. 23 shows a safety study of 10⁷ CFU LH1 delivered intranasally to healthy BALB/c mice resulting in 100% survival after 5-days.

### DETAILED DESCRIPTION

The invention provides methods and compositions for treatment of lung conditions using modified microbial strains, such as chimeric microbial hybrids and/or microbial mutants produced using selective pressure, as live biotherapeutics. For example, a modified microbe may be produced as described in U.S. Pat. No. 9,765,358, which is incorporated by reference herein in its entirety. For example, a modified microbe of GRAS (Generally Recognized as Safe) species, such as Bacillus subtilis and Lactobacillus delbrueckii sspdelbruickii, e.g., a chimeric microbial hybrid of these two species or microbial mutant(s) of one or both of these species, may be used in the methods described herein. The modified microbe may possess characteristics such as ability to: degrade a biofilm, such as a Pseudomonas biofilm; degrade mucin; competitively inhibit Staphylococcus aureus; and/or secrete a bacteriocin directed against Pseudomonas aeruginosa. In some embodiments, the live biotherapeutic may be delivered either by nasal and/or inhaled routes.

Live biotherapeutics are rationally designed to treat infectious and inflammatory diseases of the lung and/or nasal sinuses.

Safe probiotic microbes are screened for specific characteristics vital to preventing the symptoms and/or root causes of the disease, such as a microbial infection, and then survivability is improved by production of modified microbial strains. The resulting strains retain all the characteristics vital to preventing and/or treating symptoms and/or root causes, such as anti-bacterial characteristics, while being better able to survive the lung environment. Selective pressures used to produce the modified microbes described herein may include an antifungal substance, an organic compound, a solvent, high temperature, low temperature, ultraviolet light, an osmotic stressor, an inorganic chemical, ionizing radiation, composition of atmospheric gas, a vitamin or co-factor, absence of a vitamin or co-factor, an acid, a base, a carbohydrate source, a nitrogen source, a biological toxin, a peptide, a preservative substance, an herbicide, a fungicide, a pesticide, or a filtrate of another microbe's spent fermentation broth.

Modified microbial strains as described herein may work to reduce inflammation, maintain anaerobic/microaerophilic bactericidal activity towards lung pathogens, expand metabolic properties, and/or produce enzymes with activity against biofilm, DNA, and scar tissue. In some embodiments, treatment with live biotherapeutics may also interfere with cancer virulence mechanisms to support inclusion of lung cancer patients in treatment.

Unless defined otherwise herein, all technical and scientific terms used herein have the same meaning as commonly understood by one of ordinary skill in the art to which this invention belongs. Singleton, et al., Dictionary of Microbiology and Molecular Biology, second ed., John Wiley and Sons, New York (1994), and Hale & Markham, The Harper Collins Dictionary of Biology, Harper Perennial, N.Y. (1991) provide one of skill with a general dictionary of many of the terms used in this invention. Any methods and materials similar or equivalent to those described herein can be used in the practice or testing of the present invention.

Numeric ranges provided herein are inclusive of the numbers defining the range. Unless otherwise indicated, nucleic acids are written left to right in 5′ to 3′ orientation; amino acid sequences are written left to right in amino to carboxy orientation, respectively.

### Definitions

“A,” “an” and “the” include plural references (e.g., one or more) unless the context clearly dictates otherwise.

An “auxotroph” is an organism or cell that is capable of producing a nutrient required for growth. In the examples within it usually refers to the inability of a cell to make an essential amino acid(s).

The abbreviation “CFU” refers to colony forming unit.

A “chimeric organism” or “chimera” refers to a single-celled organism that contains genetic information from two or more microbial species.

“Conjugation” is the transfer of genetic material between microbes by direct cell-to-cell contact or by a bridge-like connection between two cells.

The term “culturing” refers to growing a population of cells, e.g., microbial cells, under suitable conditions for growth, in a liquid or solid medium.

The abbreviation “DMSO” refers to dimethyl sulfoxide.

The term “derived from” generally indicates that one specified material finds its origin in another specified material or has features that can be described with reference to another specified material.

The term “dosage form” refers to physically discrete units suitable as a unitary dosage for an individual to whom administered, each unit containing a predetermined quantity of active material calculated to produce the desired therapeutic or prophylactic effect, in association with a suitable pharmaceutical excipient. A “unit dose” is an amount of a substance sufficient to provide a therapeutically or prophylactically effective level in the individual for an amount of time.

An “environmental stressor” is a factor in a cell's surroundings that threatens homeostasis.

“Genotype” is the genetic constitution of an organism.

“Homeostasis” is the tendency toward a relatively stable equilibrium between interdependent elements, especially as maintained by physiological processes.

“Horizontal gene transfer” is the transmission of DNA between different species' genomes.

“Hybrid” is an organism bred from two genetically distinct varieties, species, or genera.

An “individual” or “subject” refers to a vertebrate, typically a mammal, such as a human. The term “individual” or “subject” also refers to non-human mammals, such as, for example, dogs, cats, rodents, etc.

“Live biotherapeutic” refers to live microorganisms that are applicable to the prevention, treatment, or cure of a disease or condition of human beings, animals, or plants.

A “lung condition” refers to a disease or an acute or chronic adverse condition of the lung or greater respiratory tract, including, but not limited to, a microbial infection, an inflammatory disease, and cancer.

The abbreviation “MIC” refers to minimum inhibitory concentration.

A “microbe” or “microbial strain” refers to a single-celled organism such as a bacterium or fungal cell, for example, yeast.

The term “modified” refers to the process of modifying the phenotypic expression of genes or the sequence of the underlying genomic DNA using one or more environmental stressor(s).

A “modified microbe” or “modified microbial strain” refers to a single-celled organism with one or more alteration(s) in genomic nucleotide sequence(s) or phenotypic gene expression, in comparison to the parent organism from which it was derived, induced by application of one or more environmental stressor(s), without use of recombinant technology or addition of nucleic acid from an exogenous source, such as a vector.

A “mutant” refers to an organism with one or more alteration(s) in genomic nucleotide sequence(s) in comparison to the parent organism from which it was derived.

“Oligo-sporogenic” is a microbial strain in which only a few members of the colony form spores.

An “organic compound” is any member of a large class of gaseous, liquid, or solid chemical compounds whose molecules contain carbon.

An “osmotic stressor” is a factor in the environment that causes a change in the solute concentration around a cell, causing a rapid change in the movement of water across its cell membrane.

“Parent strain” refers to a microbial strain from which a chimera or mutant is derived.

The “parental donor” is the strain contributing to genetic alterations in the parental host, either directly by providing the genetic material transferred to another species or strain through conjugation, transformation, or other DNA transfer process; or indirectly by providing the conditions, stressors, chemical inducers, etc., that facilitate DNA and/or phenotype alterations in the parental host.

The “parental host” is the strain whose DNA is altered, either through the action of environmental stressors or by receiving exogenous DNA from another species or strain through conjugation, transformation, or other DNA transfer process.

“Pharmaceutically acceptable” means approved by a regulatory agency of the Federal or a state government or listed in the U.S. Pharmacopoeia or other generally recognized pharmacopoeia for use in animals, and more particularly in humans.

“Pharmaceutically acceptable vehicle” or “pharmaceutically acceptable excipient” refers to a diluent, adjuvant, excipient or carrier with which a live biotherapeutic (modified microbe) as described herein is administered.

“Phenotype” is the observable characteristics of an organism, dependent upon genotype and environment.

“Phenotypic plasticity” is the ability of one genotype to produce more than one phenotype when exposed to different environments. Phenotypic plasticity is the ability of an organism to change its phenotype in response to changes in the environment.

A “plasmid” is an extra-chromosomal genetic element found among various strains of bacteria.

“Polyploidy” is a condition in which an organism acquires one or more additional sets of chromosomes.

“Preventing” or “prevention” refers to a reduction in risk of acquiring a disease or disorder (i.e., causing at least one of the clinical symptoms of the disease not to develop in a subject that may be exposed to or predisposed to the disease but does not yet experience or display symptoms of the disease, or causing the symptom to develop with less severity than in absence of the treatment). “Prevention” or “prophylaxis” may refer to delaying the onset of the disease or disorder.

“Prophylactically effective amount” means the amount of a modified microbe as described herein, that when administered to an individual for prevention of a disease or condition, is sufficient to effect such prevention of the disease or condition or to prevent development of at least one symptom of the disease or condition or effect development of the symptom at a lower level of severity than in the absence of administration of the compound. The “prophylactically effective amount” can vary depending on the compound, the disease and its severity, and the age, weight, etc., of the subject to be treated.

“Prophylaxis” means a measure taken for the prevention of a disease or condition or at least one symptom thereof.

A “prototroph” is an organism or cell capable of synthesizing a required nutrient. In the examples within it usually refers to the auto-synthesis of essential amino acids.

“Recombinant DNA (rDNA) molecules” are DNA molecules formed by laboratory methods of genetic recombination (such as molecular cloning) to bring together genetic material from multiple sources, creating sequences that would not otherwise be found in the genome.

The term, “recombination” is the process or act of exchanges of genes between chromosomes, resulting in a different genetic combination and ultimately to the formation of unique offspring with chromosomes that are different from those in parents.

“Strain alteration” is the addition or deletion of DNA through natural or artificial means to change gene expression in a species.

“Survivability” as used herein refers to the ability of a microbe to persist in a given environment, i.e., to maintain existence in a given location and able to be recovered from the location for outgrowth elsewhere. The microbe does not have to be actively dividing or metabolically active. For example, in the context of a lung, as described herein, if the lung were harvested, the microbe could be grown from the lung sample.

“Therapeutically effective amount” means the amount of a modified microbe as described herein, that when administered to an individual for treating a disease or condition, is sufficient to affect such treatment for the disease or condition or to reduce severity of or eliminate at least one symptom of the disease or condition. “Therapeutically effective amount” means that amount of the modified microbe that will elicit the biological or medical response of a subject that is being sought by a medical doctor or other clinician. The “therapeutically effective amount” can vary depending on the compound, the disease and its severity, and the age, weight, etc., of the subject to be treated.

A “transposon” is a chromosomal segment that can undergo transposition, especially a segment of bacterial DNA that can be translocated as a whole between chromosomal, phage, and plasmid DNA in the absence of a complementary sequence in the host DNA.

“Treating” or “treatment” of any disease or disorder refers, in one embodiment, to ameliorating the disease or disorder (i.e., arresting or reducing the development of the disease or at least one of the clinical symptoms thereof). In another embodiment “treating” or “treatment” refers to ameliorating at least one physical parameter, which may not be discernible by the subject. In yet another embodiment, “treating” or “treatment” refers to modulating the disease or disorder, either physically (e.g., stabilization of a discernible symptom), physiologically (e.g., stabilization of a physical parameter), or both.

A “vector” is a DNA molecule used as a vehicle to artificially carry foreign genetic material into another cell, where it can be replicated and/or expressed. A vector containing foreign DNA is termed recombinant DNA.

“Wild-type” refers to a microorganism as it occurs in nature.

### Methods of Treatment

Methods are provided for the treatment of lung conditions, e.g., inflammatory, infectious and fibrotic associated lung and/or greater respiratory tract diseases or chronic conditions, using modified microbes. As part of the modification process, one or a series of environmental pressure and selection tools are applied to parental microbes in order to prompt the modification of phenotypic expression or alteration in genomic nucleotide sequence(s). In one embodiment, the modified microbe is a chimeric microbial hybrid. In another embodiment, the modified microbe is a microbial mutant. Non-limiting examples of environmental pressure and selection tools that may be employed to produce modified microbes as described herein include an antifungal substance, an organic compound, a solvent, high temperature, low temperature, ultraviolet light, an osmotic stressor, an inorganic chemical, ionizing radiation, composition of atmospheric gas, a vitamin or co-factor, absence of a vitamin or co-factor, an acid, a base, a carbohydrate source, a nitrogen source, a biological toxin, a peptide, a preservative substance, an herbicide, a fungicide, a pesticide, or a filtrate of another microbe's spent fermentation broth. These modified microbes are then screened for desired traits. In this case, the desired traits include properties that allow the modified microbes to treat diseases of lung (e.g., pulmonary or respiratory) origin. These traits include among others improved survivability in the lung, the ability to kill targeted bacteria, adherence to epithelial cells and/or mucus, the ability to inhibit inflammation, the ability to remove bacterial biofilms, and the ability to exhibit mucolytic activity. Non-limiting methods for production of modified microbes may be found in U.S. Pat. No. 9,765,358, which is incorporated herein by reference in its entirety, and in the Examples below.

Examples of parent microbes that include properties that are beneficial for treatment of diseases of lung origin include species of Lactobacillus, Pediococcus, Streptococcus, Lactococcus, Leuconostoc, Oenococcus, Weissella, Bifidobacterium, Bdellovibrio, Micavibrio, Vampirovibrio, Vampirococcus, Daptobacter, Lysobacter, Myxococcus, Aristabacter, Cytophaga, Gardnerella, Clostridium, Deinococcus, Faecalibacterium, Anaerobacter, Caprobacillus, Oxobacter, Sporobacter, Eubacterium, Heliobacterium, Ocillospira, Peptococcus, Dehalobacter, Butyrivibrio, Coprococcus, Lachnospira, Ruminococcus, Peptostroptococcus, Moorella, Listeria, Mycoplasma, Bacillus, Paenibacillus, Staphylococcus, Enterococcus, Enterobacter, Escherichia, Salmonella, Klebsiella, Pseudomonas, Vibrio, Helicobacter, Haemophilus, Halomonas, Bacteroides, Prevotella, Bartonella, Porphyromonas, Actinomyces, Streptomyces, Corynebacterium, Propionibacterium, Mycobacterium, Caulobacter, Bradyrhizobium, Agrobacterium, Rhodobacter, Rhodopseudomonas, Magnetospirillum, Magnetobacterium, Acetobacter, Zymomonas, Rikettsia, Eleftheria, Saccharomyces, Schizosacchoromyces, Schefferomyces, Zygosaccharmomyces, Yarrowia, Pichia, Dekkera, Kluyveromyces, Candida, Metschnikowia, and Torulaspora. In one embodiment, the bacterial strain is a Lactobacillus species, for example, L. plantarum, L. delbrueckii, L. acidophilus, L. brevis, L. casei, L. sanfransciscensis, L. rahamnosus, L. helveticus, L. curvatus, L. sakei, L. buchneri, L. fermentum, or L. reuteri.

Examples of parent microbes that include properties that are beneficial for improved survivability in the lung include microbes from the phyla Firmicutes, Bacteroidetes, Proteobacteria, Actinobacteria, Fusobacteria, and Cyanobacteria, and more specifically species of Bifidobacterium, Gardnerella, Clostridium, Deionococcus, Faecalibacterium, Anaerobacter, Coprobacillus, Oxobacter, Sporobacter, Eubacterium, Heliobacterium, Oscillospira, Peptococcus, Dehalobacter, Butyrivibrio, Coprococcus, Lachnospira, Ruminococcus, Bacteroides, Prevotella, Bartonella, Bdellovibrio, Micavibrio, Vampirovibrio, Vampirococcus, Daptobacter, Lysobacter, Myxococcus, Aristabacter, Cytophaga, Bacillus, Paenibacillus, Staphylococcus, Enterococcus, Enterobacter, and Escherichia.

Modified microbes as described herein can be administered to an individual to treat, prevent, or ameliorate at least one symptom of a lung condition. For example, modified microbes in solid, liquid, or semi-solid form may be used to treat or prevent lung conditions in humans or non-human mammals.

Examples of lung conditions for which methods of treatment as described herein may be therapeutically or prophylactically beneficial include lung infections, inflammatory diseases, and cancer. Lung infections can be caused by bacteria, viruses, parasites and/or fungi. Examples of lung diseases caused by bacterial infections that can be treated or prevented by modified microbes include but are not limited to: pneumonia caused by Pseudomonas aeruginosa, Staphylococcus aureus, Moraxella catarrhalis, Streptococcus pyogenes, Neisseria meningitidis, Klebsiella pneumoniae, Streptococcus pneumonia, Chlamydophilia pneumonia, and Legionella pneumonophilia. Examples of symptoms associated with lung bacterial infections that can be reduced or eliminated using modified microbes include, but are not limited to: chest pain, stomach pain, fever, headaches, loss of appetite, vomiting, coughing, shortness of breath, and mucus production.

Modified microbes as described herein can be used to treat or prevent inflammatory diseases of the lung. The human lung contains a plethora of microbes. For the most part, these microbes play a beneficial role in the health of the host; however, disturbances to the abundance, diversity, and/or composition of the lung microbe community can be detrimental and cause a multitude of diseases, disorders, and/or syndromes. Examples of inflammatory diseases of the lung that can be treated or prevented using modified microbes include but are not limited to: asthma, COPD, bronchiectasis, and pneumonia.

Modified microbes as described herein can be used to treat or prevent other diseases or syndromes associated with or originating in the lung. Examples of such diseases include, but are not limited to: metabolic disorder, autoimmune disease such as rheumatoid arthritis, lupus, sarcoidosis, scleroderma or Sjogren's syndrome, fibrotic disease (FD) such as cystic fibrosis (CF), idiopathic pulmonary fibrosis (IPF), interstitial pneumonia, and cancer (e.g., adenocarcinoma, squamous cell carcinoma, or large cell carcinoma).

Modified microbes as described herein can be administered to subjects in various ways. For example, modified microbes in solid, liquid, or semi-solid form, e.g., as an inhaled solution or powder, nasal spray, or nasal/lung irrigation solution, can be administered to an individual. For example, inhaled delivery may occur once, twice, thrice, or four times per day for 1 to 21 days or longer, e.g., 1, 2, 3, 4, 5, 6, 7, 10, 14, or 21 days.

Modified microbes as described herein may be administered either singly or as mixture of multiple microbes. Dosage may vary based on particular modified microbe(s) used, specific ailment to be treated, age, weight, and health of the patient, the patient's response to treatment, and/or other parameters, which may be evaluated by a physician or other medical professional. Example dosages of modified microbes, which may be delivered by inhalation, intranasally, and/or intratracheal installation/irrigation, include about 10 to about 10¹² or more, e.g., about 10, about 100, about 1000, about 10⁴, about 10⁵, about 10⁶, about 10⁷, about 10⁸, about 10⁹, about 10¹⁰, about 10¹¹, or about 10¹², or more microbes in a single dose. For example, any of about 10 to about 100, about 100 to about 1000, about 10⁴ to about 10⁵, about 10⁵ to about 10⁶, about 10⁶ to about 10⁷, about 10⁷ to about 10⁸, about 10⁸ to about 10⁹, about 10⁹ to about 10¹⁰, about 10¹⁰ to about 10¹¹, about 10¹¹ to about 10¹², about 10 to about 1000, about 100 to about 10⁴, about 1000 to about 10⁵, about 10⁴ to about 10⁶, about 10⁵ to about 10⁷, about 10⁶ to about 10⁸, about 10⁷ to about 10⁹, about 10⁸ to about 10¹⁰, about 10⁹ to about 10¹, about 10¹⁰ to about 10¹², about 10 to about 1000, about 100 to about 10⁵, about 1000 to about 10, about 10⁵ to about 10⁸, about 10⁶ to about 10⁹, about 10⁹⁷ to about 10¹⁰, about 10⁸ to about 10¹¹, about 10⁹ to about 10¹², about 10 to about 10⁵, about 1000 to about 10⁸, or about 10⁶ to about 10¹² or more.

Modified microbes described herein may be used in combination with other therapeutic agents to treat lung conditions. Examples of other therapeutic agents that may be used in combination with modified microbes include, but are not limited to, antibiotics, antifungals, bacteriophage, peptides, enzymes, sugars, glycopolymers, bacteriocins, probiotics, antibodies, and spores. For example, a synergistic or additive effect may be achieved by administering modified microbes in combination with one or more additional therapeutic agent(s), either concurrently or sequentially. An additional therapeutic agent may be administered prior to, concurrently with, or after administration of modified microbe.

In some embodiments, an additional therapeutic agent may be administered prior to modified microbes. An example of this would be to administer one or more antibiotic (e.g., amoxicillin and clavulanic acid, cloxacillin and clavulanic acid, cloxacillin and dicloxacillin, cephalexin, cefdinir, cefprozil, cefaclor, cefuroxime, sulfamethoxazole and trimethoprim, erythromycin/sulfisoxazol, erythromycin, clarithromycin, azithromycin, tetracycline, doxycycline, minocycline, tigecycline, vancomycin, imipenem, meripenem, clistimethate/colistin, methicillin, oxacillin, and nafcillin, cabenicillin, ticarcillin, piperacillin, mezlocillin, and azlocillin, ticarcillin and clavulanic acid, piperacillin and tazobactam, cephalexin, cefdinir, cefprozil, and cefaclor, cefepime, tobramycin, amikacin, gentamicin, clarithromycin, and azithromycin, ciprofloxacin, levofloxacin, aztreonam, and linezolid) or a bacteriophage to clear out unwanted microbes from the lung, followed by one or more doses of modified microbe to prevent further infections. Another example would be to administer an antibiotic such as tobramycin to clear a primary infection of a microbial infection, such as P. aeruginosa, followed by one or more doses of modified microbes to prevent the establishment of chronicity of the infection. In some embodiments, an additional therapeutic agent may be administered contemporaneously with modified microbe administration.

### Compositions

Compositions are provided that include one or more modified microbe as described herein. In some embodiments, the composition is a pharmaceutical composition, and includes at least one pharmaceutically acceptable excipient. In some embodiments, the composition includes a carrier molecule.

In some embodiments, the composition is formulated with a motile organism (or otherwise optimized for targeting of infection) for delivery to a desired site of action within an individual to whom it is administered. For example, the composition may be formulated for nasal administration.

In some embodiments, the composition is formulated for delivery to a desired site of action within an individual to whom it is administered. For example, the composition may be formulated for administration to the large and small airways in the lung, or in the bronchioles.

When employed as pharmaceuticals, i.e., for treatment or prophylaxis of a lung condition, the compositions described herein are typically administered in the form of a pharmaceutical composition. Such compositions can be prepared in a manner well known in the pharmaceutical art and include at least one active compound, i.e., a modified microbe as described herein.

Generally, the compositions are administered in a pharmaceutically effective amount, i.e., a therapeutically or prophylactically effective amount. The amount of the active agent, i.e., a modified microbe as described herein, actually administered will typically be determined by a physician, in the light of the relevant circumstances, including the condition to be treated, the chosen route of administration, the activity of the modified microbe administered, the age, weight, and response of the individual patient, the severity of the patient's symptoms, and the like.

The pharmaceutical compositions can be administered by a variety of routes including inhalation, intranasal, or intratracheal instillation. Depending on the intended route of delivery, the pharmaceutical compositions are preferably formulated as dry powder compositions or solutions.

The compositions for inhaled administration can take the form of bulk liquid solutions or suspensions, or bulk powders. More commonly, however, the compositions are presented in unit dosage forms to facilitate accurate dosing. Typical unit dosage forms include prefilled, premeasured ampules or syringes of the liquid compositions, gelatin capsule, foil-foil blister, or metered drug reservoir or the like in the case of solid compositions. In some embodiments of such compositions, active agent, i.e., a modified microbe as described herein, may be a minor component (about 0.1% to about 50% by weight, or about 1% to about 40% by weight) with the remainder being various vehicles or carriers and processing aids helpful for forming the desired dosing form.

Liquid forms suitable for inhaled administration may include a suitable aqueous or nonaqueous vehicle with buffers, suspending and dispensing agents, colorants, flavors and the like. Solid forms may include, for example, any of the following ingredients, or compounds of a similar nature: an excipient such as galactose or mannose.

In some embodiments, for formulation into dosage forms for use in the methods described herein, the modified microbes can be lyophilized (e.g., freeze-dried) and then blended together in powder form with other ingredients in order to increase protein production or survivability, extend shelf life, and optimize product parameters. In some embodiments, these ingredients include: water; salts; sugar, such as monosaccharides (e.g., glucose, fructose, galactose, mannose, arabinose, xylose), disaccharides (e.g., sucrose, lactose, cellobiose, maltose, trehalose), trisaccharides (e.g., raffinose, melezitose, maltotriose), and/or oligosaccharides (e.g., starch, glycogen, cellulose, xylan, fructooligosaccharide, inulin); sugar alcohols (e.g., xylitol, mannitol, sorbitol, glycerol).

The above-described components for pharmaceutical compositions are merely representative. Other materials as well as processing techniques and the like are set forth in Part 8 of Remington's The Science and Practice of Pharmacy, 21st edition, 2005, Publisher: Lippincott Williams & Wilkins, which is incorporated herein by reference.

### Kits

Kits are provided for use in methods of treatment of lung conditions as described herein. For example, a kit may include one or more unit dose of modified microbes as described herein. The modified microbes may be formulated in a pharmaceutical composition, e.g., in one or more therapeutically or prophylactically effective amount for the lung condition to be treated, e.g., in one or more dosage form. Optionally, instructions for use and/or administration, e.g., inhaled or intranasal administration, of the composition, in a method described herein, are provided.

Instructions for a kit as described herein may be provided in printed form or in the form of an electronic medium such as a CD or DVD, or in the form of a website address where such instructions may be obtained or a mobile application.

A kit may be provided in suitable packaging. As used herein, “packaging” refers to a solid matrix or material customarily used in a system and capable of holding within fixed limits a composition suitable for use in a method as described herein. Such materials include glass and plastic (e.g., polyethylene, polypropylene, and polycarbonate) bottles, vials, paper, plastic, and plastic-foil laminated envelopes and the like. If e-beam sterilization techniques are employed, the packaging should have sufficiently low density to permit sterilization of the contents.

The following examples are intended to illustrate, but not limit, the invention.

### EXAMPLES

### Example 1

### Modified Microbes with Therapeutic Traits for Treatment of Lung Infections with Multiple Modes of Action

Modified microbes were produced using methods previously described (U.S. Pat. No. 9,765,358). The modified microbe LH1 originated from a process that applied metabolic (protein) and atmospheric (oxygen) stressors on Lactobacillus delbrueckii and Bacillus. subtilis, respectively. This resulted in LH1, which possessed the ability to metabolize 8 new carbohydrates while retaining anaerobic respiration.

Briefly, parent cultures (B. subtilis and LH1) were mixed together and filtered on to a sterile nitrocellulose 0.2 μm filter. Filters were then washed via syringe with 10 ml of sterile PBS. Filters were placed upside down on to a deoxygenated, M9 alginate plate. Plates were incubated inside an anaerobic chamber at 37° C. for 72 hours.

After incubation, filters from each condition were placed into 1 ml of PBS inside a 1.5 ml microcentrifuge tube. Microcentrifuge tubes containing the filters were vortexed and then spun down at 8000 RFC for 10 minutes. The cell pellet was resuspended in PBS and spread on to two fresh, anaerobic M9 NB plates. Plates were incubated in an anaerobic chamber for 96 hours. No colonies were seen, so plates were moved to an aerobic incubator for 16 hours to germinate any spores.

Plates were checked for colonies after 16 hours of aerobic incubation. Single colonies were then re-streaked on to M9 NB aerobic plates and moved into an anaerobic chamber after 4 hours (the B. subtilis parent requires 4 hours of oxygen exposure to germinate from spore form). Plates were examined after 24 to 120 hours of anaerobic incubation for colonies.

Ten colonies grew within 48 hours. All modified microbes were analyzed for bacteriocin activity against Pseudomonas, anaerobic growth, carbohydrate metabolism profile, alginate lyase production, proteinase and mucinase production. Three of these ten modified microbes could double four to eight times before sporulating (F2BH1, F2BH2, F2BH3) and two of these strains also acquired the bacteriocin against Pseudomonas (F2BH2, F2BH3).

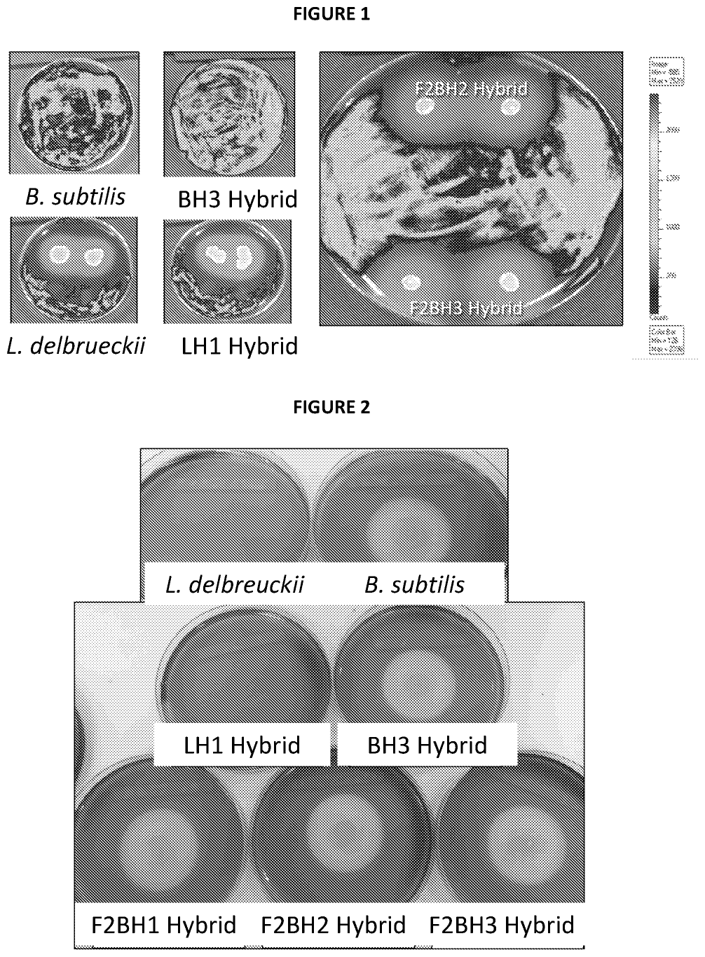

FIG. 1 shows F2BH2 (top 2 patches) and F2BH3 (bottom 2 patches) as white patches with clearance from anaerobic bacteriocin activity at 48 hours against a luciferase producing strain of Psuedomonas aeruginosa spread on the plate; pictures from bioluminescence in an IVIS machine. The wildtype B. subtilis parent showed no anaerobic activity against Pseudomonas compared to the wildtype L. delbrueckii parent whose white patches and clearance show the bacteriocin activity. The modified microbe BH3 originated from a process that applied metabolic (protein) and atmospheric (oxygen) stressors to L. delbrueckii and B. subtilis, respectively. This resulted in BHS, which possessed the ability to metabolize one new carbohydrate while retaining aerobic respiration, alginate lyase, and mucinase activity. This modified microbe, BH3, showed minimal anaerobic activity against Pseudomonas (4 small dots of clearance), compared to LH1 which retained the bactericidal activity of its L. delbrueckii parent.

A volume of 20 μl of overnight aerobic cultures grown in modified BHI were dropped on the sodium alginate plates. FIG. 2 depicts alginate lyase production using plates containing sodium alginate which have been stained with iodine. Iodine binds sodium alginate and shows clearance zones where it has been degraded by alginate lyase. No alginate lyase activity was observed from the wildtype Lactobacillus parent but alginate lyase production was present in the wildtype Bacillus parent. After the first round of modification, one modified microbe (LH1) was generated and showed no alginate lyase activity. Another modified microbe (BH3) retained alginate lyase production. Alginate lyase production was observed in all three secondary modified microbes, F2BH1, F2BH2, and F2BH3.

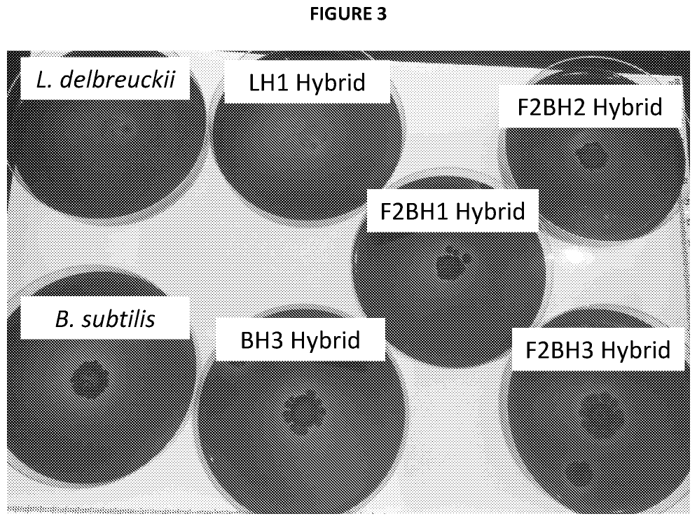

Strains were grown overnight in modified BHI aerobically and 20 μl was dropped in the center of 0.3% mucin plates. Plates were soaked for in 0.1% amido black in 3.5 M acetic acid for 30 min, and then washed with 1.2 M acetic acid. Mucin lysis zone (discolored halo) around the colonies was observed after 24 hours of aerobic incubation at 37° C. FIG. 3 shows the wildtype Bacillus, BH3, and all three secondary modified microbes, F2BH1-3 show mucin degradation.

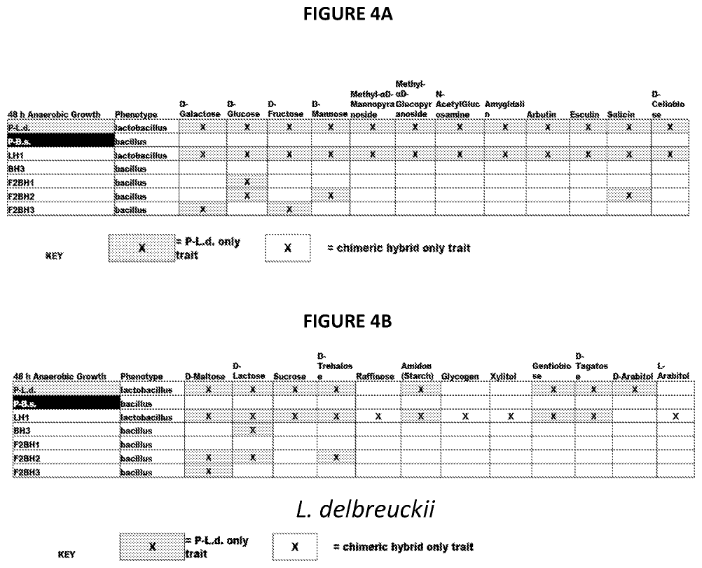

Strains were tested for anaerobic carbohydrate metabolism using Biomerieux's api 50 CH test following 48 hours of anaerobic incubation at 37° C. As seen in FIG. 4, the wildtype Bacillus parent showed no carbohydrate metabolism in anaerobic conditions. BH3 showed only metabolism of lactose and F2BH1-3 showed varying levels of anaerobic carbohydrate metabolism with F2BH2 demonstrating the largest number of carbohydrates metabolized for a morphological B. subtilis modified microbe in anaerobic conditions.

### Example 2

### Live Biotherapeutics Demonstrate Antimicrobial Activity Against Pathogenic Organisms, Including Multi-Drug Resistant Pathogens

Modified bacterial strains produced metabolites and bacteriocins with antimicrobial activity against pathogenic bacteria. Antibacterial activity was demonstrated against drug resistant pathogens in a modified cross-streak assay. For the modified cross-streak assay modified microbes (LH1 or wildtype Lactobacillus parent) were grown in BHIL broth overnight and streaked in a single line across BHIL agar acclimated to anaerobic conditions. The plate was incubated at 37° C. anaerobically for 48 hours before pathogenic strains obtained from ARBank (CDC) and control bacteria were cross-streaked perpendicular to the modified microbe and grown for 24 hours aerobically. The distance from the modified microbe growth where pathogenic bacteria growth was inhibited was measured (mm) with calipers and normalized to control.

Table I shows exemplary bacteria demonstrating sensitivity to modified microbes (LH1).

TABLE I
Exemplary bacterial with sensitivity to live biotherapeutic LH1
bacteriocins.

| | Median inhibition by LH1 | Range |
|---|---|---|
| Bacteria (n) | (mm) | (mm) |
| Escherichia coli (15) | 10 | 4.5-15 |
| Klebsiella pneumoniae (14) | 7 | 4-12 |
| Enterobacter aerogenes (3) | 11 | 9-11.5 |
| Serratia marcescens (1) | 12.5 | 12.5 |
| Klebsiella oxytoca (1) | 11 | 11 |
| Acinetobacter baumannii (29) | 13 | 10-19 |
| Pseudomonas aeruginosa (43) | 7 | 4-9 |
| Staphylococcus aureus (23) | 10 | 8-22 |
| Enterococcus avium (1) | 6.5 | 6.5 |
| Enterococcus faecium (6) | 6 | 4.5-8.5 |
| Enterobacter cloacae (2) | 11 | 10-12 |
| Burkholderia cenocepatia (2) | 8 | 4.5-12 |
| Burkholderia multivorans (2) | 13 | 11.5-14 |
| Burkholderia gladioli (2) | 12 | 11-12 |
| Stenotrophomonas maltophilia (1) | 12 | 12 |
| Achromobacter xylosoxidans (1) | 13 | 13 |
| Achromobacter dolans (1) | 12 | 12 |
| Achromobacter ruhlandii (1) | 11 | 11 |

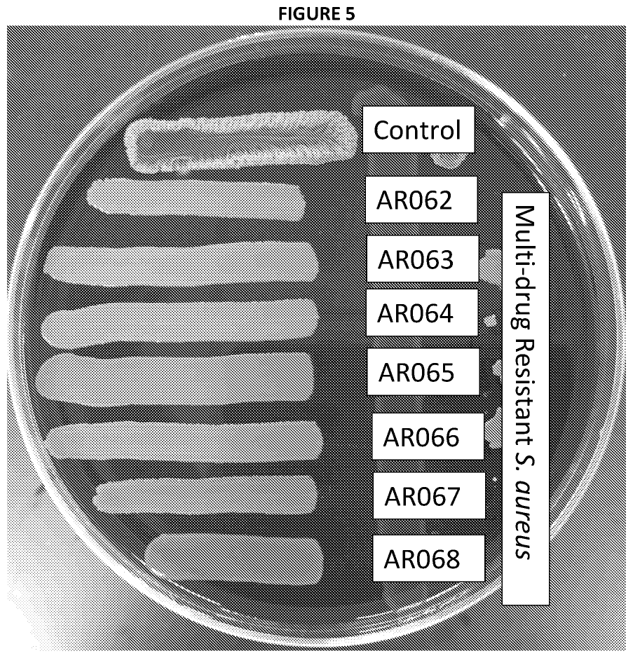

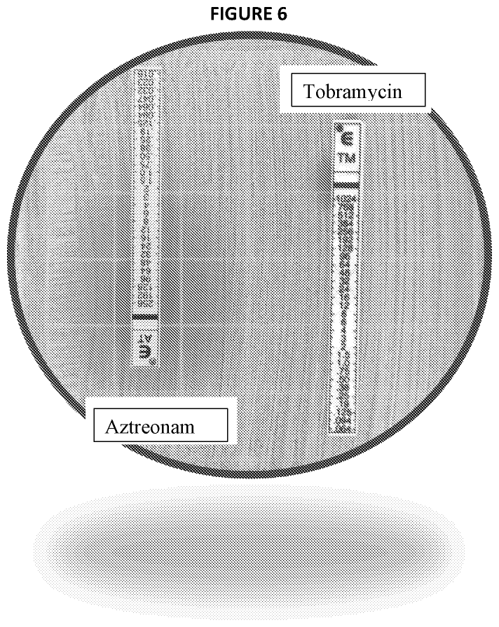

Note that the antimicrobial activity, measured by a modified cross-streak assay, was broad range in inhibiting the growth of both Gram-positive and Gram-negative bacteria known to be associated with infection or colonization contributing to disease. An example of the cross-streak assay is shown in FIG. 5 where a control strain at the top (B. methylotrophicus) is resistant to LH1 bacteriocin that has diffused through the agar after 48 hours anaerobic growth while multidrug resistant S. aureus strains from the CDC AR Bank (AR0562-AR0568) were sensitive and the distance from LH1 was measured and recorded. Table II shows LH1 ETEST® results on sensitivity, including CF-associated agents, aztreonam and tobramycin (FIG. 6).

TABLE II
Antibiotic sensitivity of LH1.

| | Susceptible | Resistant |
|---|---|---|
| Antibiotic | to (μg/ml) | to (μg/ml) |
| Clarithromycin | 0.16 | |
| Aztreonam (AT) | 2.0 | |
| Rifampicin | 0.125 | |
| Amoxicillin/Clavulanic Acid | 0.125 | |
| Piperacillan | 0.19 | |
| Linezolid | 0.5 | |
| Tetracycline | 0.5 | |
| Erythromycin | 0.023 | |
| Imipenem | 0.094 | |
| Vancomycin | 1.5 | |
| Ciprofloxacin) | | 32 |
| Amikacin | | 48 |
| Levofloxacin | | 12 |
| Trimethoprim/Sulfamethoxazole | | 32 |
| Tobramycin (TM) | | >512 |

The antimicrobial activity was broad range in inhibiting the growth of both Gram-positive and Gram-negative bacteria. Antibiotic sensitivity demonstrates innate resistance to a key antibiotic (tobramycin) that may be co-administered during lung infection, but largely susceptible to most antibiotics.

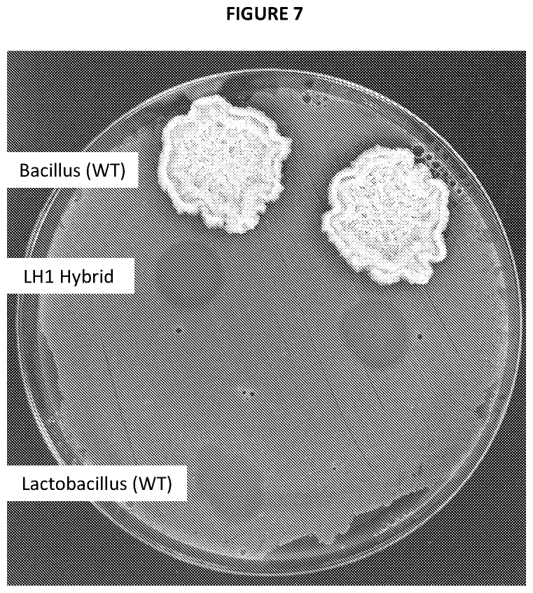

In a similar qualitative analysis, the drop test was used to demonstrate the ability of modified microbes to inhibit the growth of fluorescent P. aeruginosa. In another drop test (top two drops bacteria, bottom two drops filtered supernatant) inhibition of growth by LH1 live cells and/or filtered supernatant was demonstrated (FIG. 1). Another drop test shows the zones of inhibition of S. aureus CF clinical isolate growth by LH1 and Lactobacillus parent strain supernatant containing metabolites accumulated over 24 hours of anaerobic growth (FIG. 7). The Bacillus parent strain showed no zone of inhibition following 24 hours of aerobic growth.

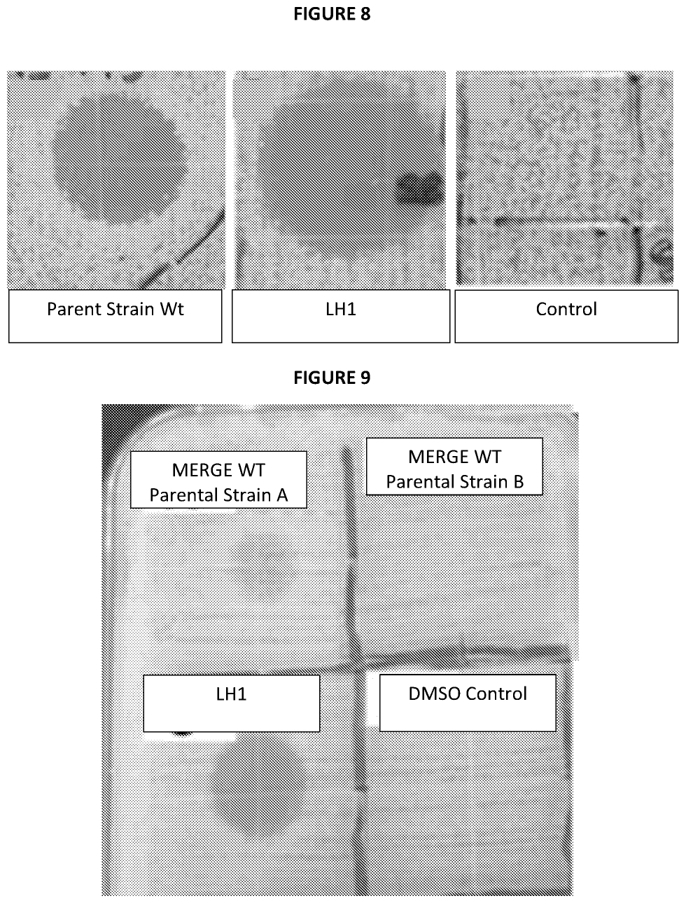

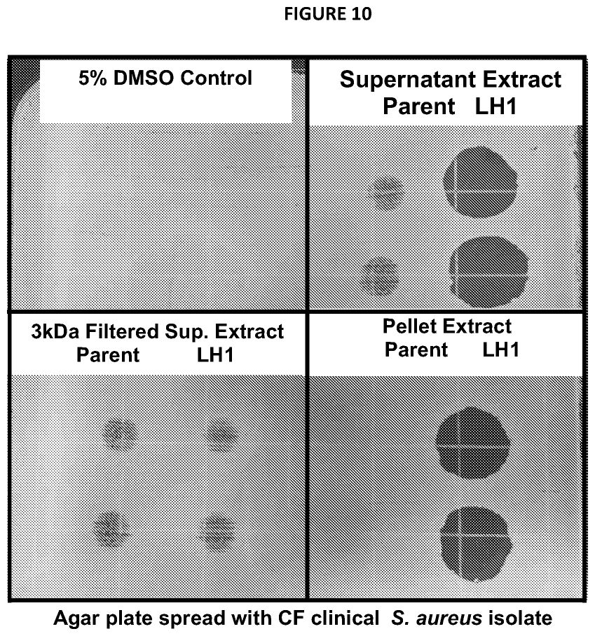

In additional drop tests whole cell crude extracts show antibacterial activity of LH1 against P. aeruginosa (FIG. 8) and S. aureus (FIG. 9) CF clinical isolates is increased in the LH1 compared to parental strains. Further drop tests of 3 kDa filtered crude extracts indicate that an approximately 10 kDa secreted protein is responsible for the increased antibacterial activity against a CF isolate of S. aureus (FIG. 10).

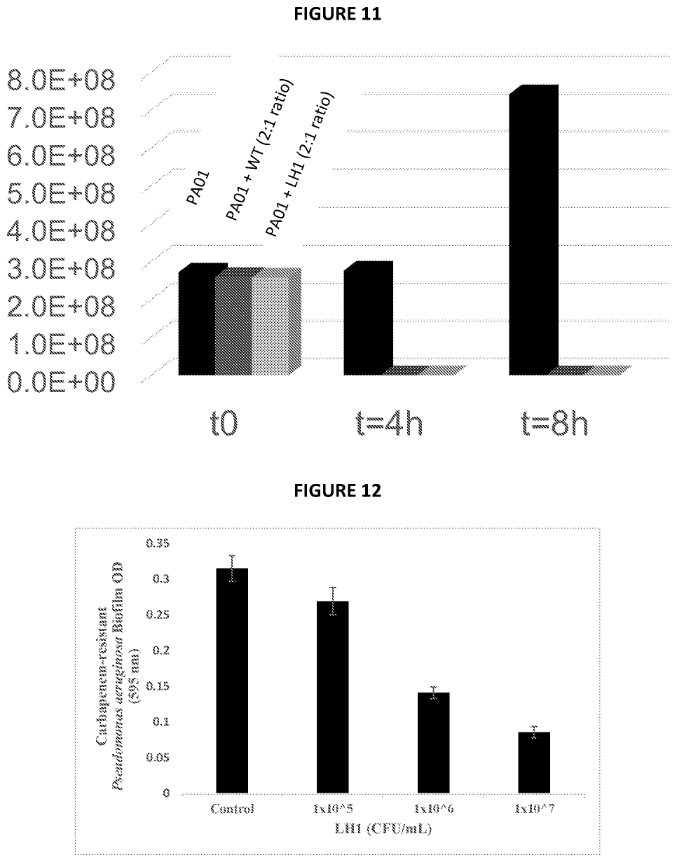

In vitro antibacterial activity was also tested in the planktonic state. P. aeruginosa strain PA01 (2×10⁸ cfu/mL) and WT parental strain (1×10⁸ cfu/mL) and LH1 (1×10⁸ cfu/mL) were both mixed in 2:1 ratio. The number of viable P. aeruginosa were determined at 4 hours and 8 hours following exposure. This showed that P. aeruginosa was killed and growth inhibited by both WT and LH1 up to 8 hours (FIG. 11).

### Example 3

### The Ability of Live Biotherapeutics to Remove Anaerobic P. aeruginosa Biofilms

The ability of modified microbes (LH1) to remove anaerobic P. aeruginosa biofilms was investigated. The carbapenem resistant P. aeruginosa clinical isolate (AR0243; CDC ARBank) culture was inoculated 1:1000 in LB broth and grown to mid-log, then centrifuged and resuspended in an equal volume of LBN media (LB with 10 g/L nitrate) and biofilms were grown statically under anaerobic conditions in 96-well tissue culture plates for 3 days at 37° C. The biofilms were washed with PBS to remove non-adherent cells, and then treated with LH1 for 4 hours. The treated biofilms were washed with PBS to remove non-adherent cells and stained with 1% crystal violet stain for 30 minutes. Following washing with PBS, ethanol was placed in the well for 5 minutes and then removed and placed in a fresh 96-well plate. The OD600 was measured to quantify the remaining biofilm.

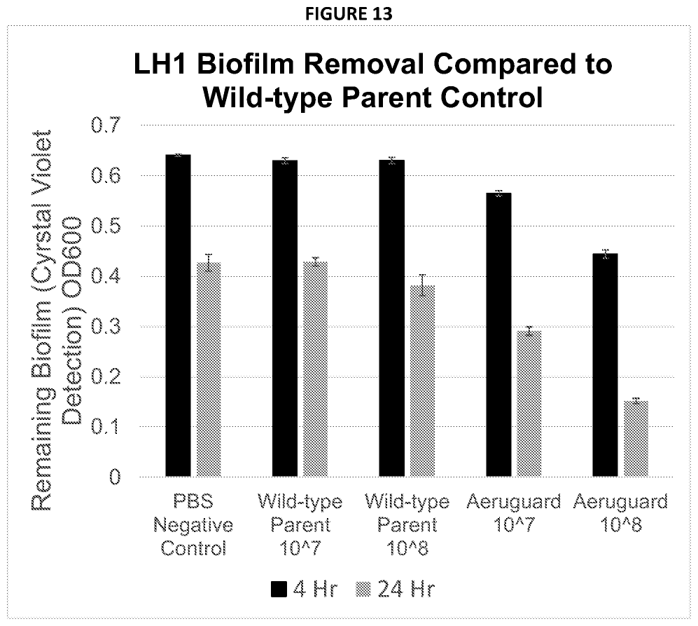

FIG. 12 shows that a modified microbe was effective in removing carbapenem resistant P. aeruginosa clinical isolate in an anaerobic environment. Treatment of the biofilms with 10⁶ and 10⁷ CFU/mL LH1 resulted in up to 70% reduction of biofilm, respectively, compared to PBS control after 4 hours. FIG. 13 shows that the reduction of P. aeruginosa biofilm by LH1 is greater than that observed by the WT parent. These data suggest that LH1 may be effective in treating multidrug-resistant pathogens during lung infections.

### Example 4

### Antibacterial Activity in CF Sputum

The growth of LH1 in CF mucin media and CF sputum. The CF mucin media was inoculated with about 10⁷ cfu/mL and plated for viable counts after 0 and 4 hours. Also, pooled sputum from 4-5 CF subjects was diluted 1:2 sputum:PBS (w/v) and homogenized by bead beating. Aliquots were inoculated with approximately 10⁵ cfu/mL LH1. Inoculated sputum was plated on non-selective media and grown anaerobically to enumerate LH1 over time.

To examine the activity of LH1 in CF sputum, pooled sputum from 4-5 CF subjects was diluted 1:2 sputum:PBS (w/v) and homogenized by bead beating. Aliquots were treated with LH1 in an anaerobic time-kill assay. Treated sputum was plated for viable counts on S. aureus selective media (Vogel-Johnson Agar).

In a similar study, ethyl acetate was used to obtain a crude extract of 100× concentrated LH1 spent media after 4 days growth. The extract was diluted into the sputum at a 1:10 ratio and incubated for up to 2 hours. The extract was also diluted and 10 μl was placed into blank antibiotic testing discs at a 1:2 and 1:100 ratio before being placed on a plate streaked for lawn growth with 0.5 MacFarland P. aeruginosa (AR0243). The plates with crude extract discs (and 5% dimethyl sulfoxide control, DMSO) were incubated overnight at 3TC.

Whole genome sequencing was used to identify the genetic variations that supported the modified antibacterial and metabolic activity of LH1. Two trackable and stable genetic modifications were identified. Primers Perm03-F (5′-GCCGCCATAAAGCAAATGATCA-3′) (SEQ ID NO:1) and Perm01-R (5′-AGCCATCATGAACCGTCTCTTC-3′) (SEQ ID NO:2) along with the OneTaq Hot Start DNA polymerase (NEBiolabs) were used to amplify the region in a permease of the drug/metabolite transporter (DMT) superfamily shown to contain one of the genetic modifications (T deletion). This PCR tool was used to validate the stability of the genetic modifications in 10 single colonies obtained after more than 60 generations of passage. PCR products from the 10 colonies were purified and sent for Sanger sequencing.

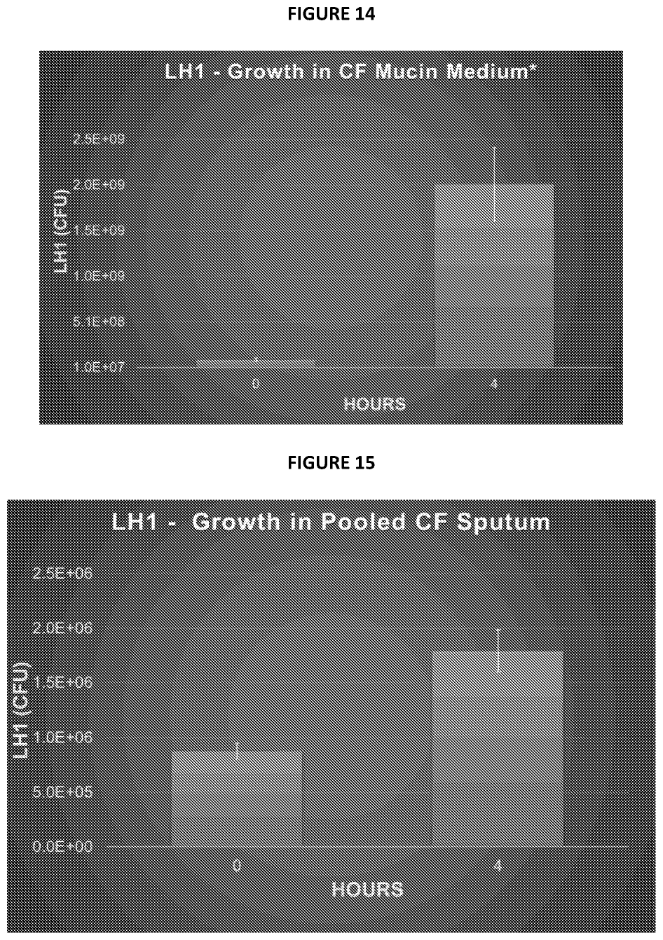

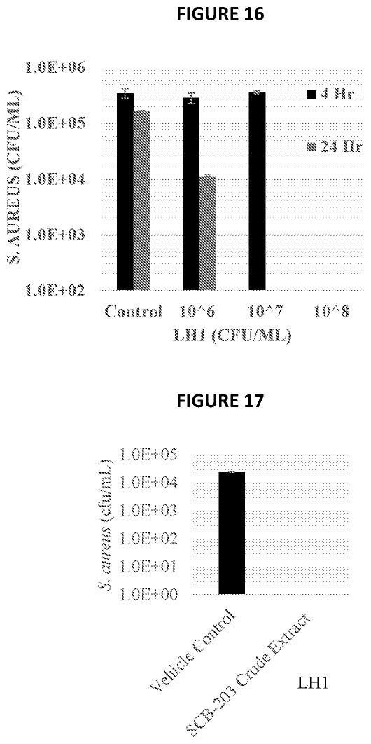

FIG. 14 shows that LH1 continues to grow in CF mucin media after 4 hours, resulting in a 20-fold increase in LH1 following inoculation with 10⁷ cfu/mL. FIG. 15 shows that LH1 continues to grow in CF sputum after 4 hours, resulting in a 2-fold increase in LH1 following inoculation with 10⁵ cfu/mL. It is noted that the antibiotics present in the CF sputum are unknown. FIG. 16 shows that LH1 reduced S. aureus in CF sputum in a time and dose dependent manner. No S. aureus was detected in sputum treated with 10⁸ CFU/mL of LH1 for 4 or 24 hours, or 10⁷ cfu/mL for 24 hours, respectively, demonstrating a >4 log reduction.

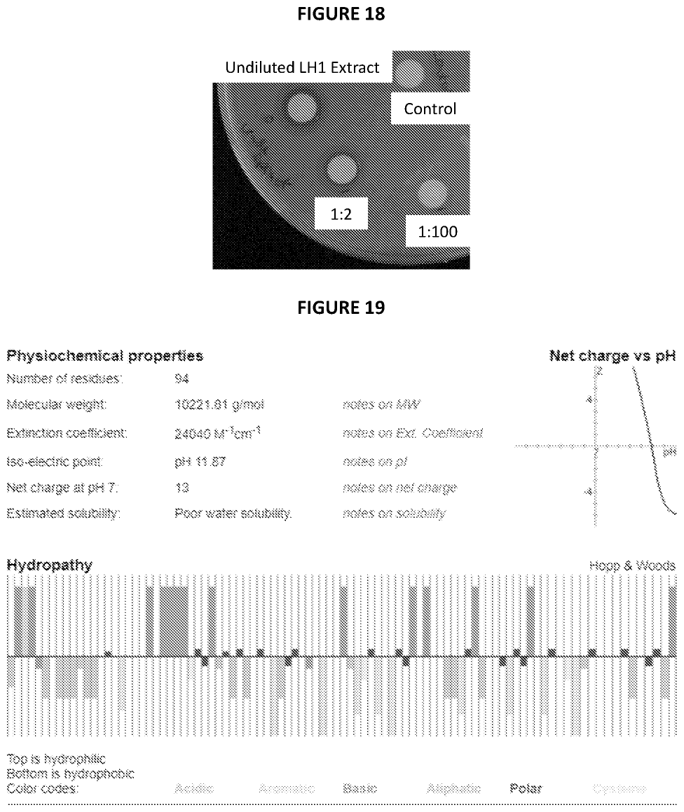

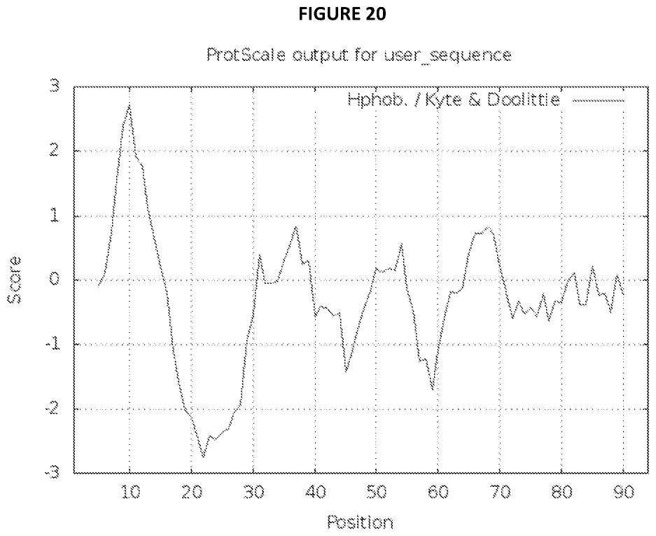

Incubation with crude extract of LH1 showed complete eradication of S. aureus (from 10⁴ to 0 cfu/mL) within 1 hour, demonstrating the potent anti-S. aureus activity of the bacteriocin produced by LH1 (FIG. 17). Further a zone of inhibition was observed around the LH1 crude extract discs compared to 5% DMSO control following overnight incubation of the P. aeruginosa lawn (FIG. 18). The zone of inhibition showed a dose dependence in that the diameter of the zone decreased upon dilution.

Whole genome sequencing showed LH1 has a deletion in a permease gene of the drug/metabolite transporter (DMT) superfamily which causes a frameshift that results in a novel protein being expressed and is responsible for the increase antibacterial activity against S. aureus. This new protein was characterized and shown to be 10.2 kDa, with a possible secretion signal peptide leader, an isoelectric point of pH 11.87, a net positive charge (13) at physiological pH and a hydropathy profile suggesting poor water solubility (FIG. 19, 20). Further characterization of the new protein sequence also indicates the presence of a possible leader sequence that may facilitate protein secretion and a net positive charge, which are attracted to and incorporated into negatively charged bacterial membranes (FIG. 19). The second gene change is a point mutation in PTS, fructose specific transporter subunit IIC; the substitution (A>G) resulted in the loss of stop codon in an intergenic region that extends the 5′ end of ORF. This may contribute to the modified metabolic activity but remains to be fully characterized.

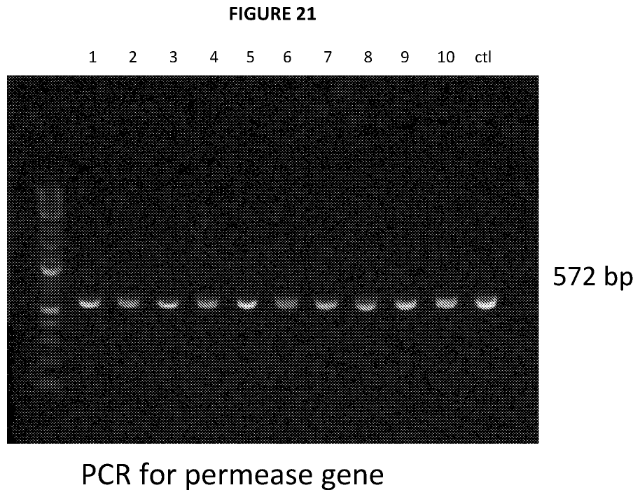

Following over 60 generations of passage, PCR was used to amplify the modified permease region of 10 individual colonies. These PCR products were sequenced and found to have retained the genetic modification suggesting the genetic changes are stable and trackable (FIG. 21).

### Example 5

### Live Biotherapeutics and Bacteriocins Demonstrated Mucolytic Activity and Reduced Mucus Viscosity

Sputum samples were obtained from seven cystic fibrosis patients during routine clinical practice, pooled, diluted 1:2 sputum:PBS (w/v), and homogenized by bead beating. A volume of 100 μL of pooled sputum was placed into microfuge tubes and treated with 10⁷-10⁸ CFU/mL live biotherapeutics (in this example LH1). After a brief mixing step, the tubes were incubated at 37° C. for 4 hours anaerobically to simulate conditions in the patient. Similarly, a volume of 70 μL of pooled sputum and 100× crude extract of LH1 or sterile PBS (1:1) were incubated anaerobically for 4 hours then tested for pourability.

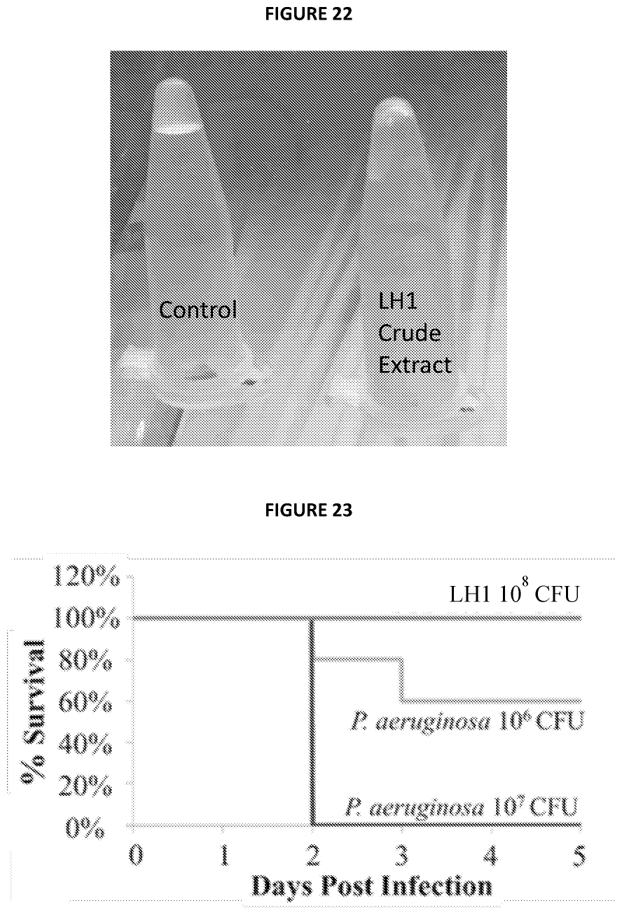

Untreated sputum samples were unable to pour, however the live biotherapeutic crude extract treated sputum was pourable as shown in FIG. 22. Treatment with live biotherapeutics (LH1) demonstrated an ability to reduce the viscosity of cystic fibrosis sputum.

### Example 6

### Live Biotherapeutic Lung Delivery does not Induce Mortality in Mice

The safety profile of live biotherapeutics (modified microbes) is an important consideration. Initial in vivo studies used a murine model to provide a preliminary evaluation. Three groups of mice were evaluated over 5 days following intranasal inoculation with either F2BH2 live biotherapeutic or P. aeruginosa at either 10⁶ or 10⁷ CFU/mL.

This acute safety study in a murine lung infection model showed F2BH2, applied intranasally to mice at 10⁸ CFU/mL, resulted in 100% survival after 5 days. Mice infected with P. aeruginosa intranasally at 10⁶ or 10⁷ CFU/mL showed 60 and 0% survival after 5 days, respectively (FIG. 23).

### Example 7

### Modified Microbes Interfere with Lung Cancer Mechanism for Immune Evasion

An in vitro cell culture method is used to examine the influence of modified microbes one the immune response to cancer. Treatment with live biotherapeutics (one or more modified microbe(s) as described herein) to evaluate the host response following treatment.

### Example 8

### Live Biotherapeutic Reduces Inflammation in the Lung and Sinuses

An animal model is used to examine biomarkers of disease and inflammation. Treatment with live biotherapeutics (one or more modified microbe(s) as described herein) to reduce inflammation and markers of disease in the lungs is evaluated following treatment in the lungs.

### Example 9

### Live Biotherapeutic Reduces Chronic Infection in the Lung and Sinuses

An animal model is used to examine the reduction of infection in the lungs of infected animals. Treatment with live biotherapeutics (one or more modified microbe(s) as described herein) to reduce infection in the lungs is evaluated following treatment in the lungs.

### Example 10

### Live Biotherapeutic Demonstrates Safety for Chronic Use in Healthy or Diseased Lung and Sinuses

An animal model is used to examine the safety of chronic use of live biotherapeutics in the lungs/sinuses of healthy and diseased animals. Treatment with live biotherapeutics (one or more modified microbe(s) as described herein) to demonstrate safe use in the lungs is evaluated following treatment in the lungs or sinuses.

### Example 11

### Live Biotherapeutic Demonstrates Influence on Lung Microbiome in Healthy or Diseased Lung and Sinuses

An animal model is used to examine the influence on the lung, sinus, and oropharynx, and GI microbiome following use of live biotherapeutics (one or more modified microbe(s) as described herein) in the lungs/sinuses of healthy and diseased animals. Treatment with live biotherapeutics to demonstrate an influence on the microbiome is evaluated following treatment in the lungs or sinuses.

Although the foregoing invention has been described in some detail by way of illustration and examples for purposes of clarity of understanding, it will be apparent to those skilled in the art that certain changes and modifications may be practiced without departing from the spirit and scope of the invention, which is delineated in the appended claims. Therefore, the description should not be construed as limiting the scope of the invention.

All publications, patents, and patent applications cited herein are hereby incorporated by reference in their entireties for all purposes and to the same extent as if each individual publication, patent, or patent application were specifically and individually indicated to be so incorporated by reference.

1. A method for treatment or prevention of a lung condition, said method comprising administering a therapeutically or prophylactically effective amount of a modified microbe to an individual in need thereof, wherein said administering results in prevention, amelioration, or elimination of at least one symptom of a lung condition in the individual.

2. A method according to claim 1, wherein said modified microbe comprises a chimeric microbial hybrid or a microbial mutant produced by application of one or more environmental pressure condition.

3. A method according to claim 1, wherein said modified microbe comprises properties that are beneficial for treatment of said lung condition and/or properties that are beneficial for improved survivability in the lung.

4. A method according claim 1, wherein said modified microbe is derived from a parent microorganism that comprises properties that are beneficial for treatment of said lung condition, selected from species of Lactobacillus, Pediococcus, Streptococcus, Lactococcus, Leuconostoc, Oenococcus, Weissella, Bifidobacterium, Bdellovibrio, Micavibrio, Vampirovibrio, Vampirococcus, Daptobacter, Lysobacter, Myxococcus, Aristabacter, Cytophaga, Gardnerella, Clostridium, Deinococcus, Faecalibacterium, Anaerobacter, Coprobacillus, Oxobacter, Sporobacter, Eubacterium, Heliobacterium, Oscillospira, Peptococcus, Dehalobacter, Butyrivibrio, Coprococcus, Lachnospira, Ruminococcus, Peptostreptococcus, Moorella, Listeria, Mycoplasma, Bacillus, Paenibacillus, Staphylococcus, Enterococcus, Enterobacter, Escherichia, Salmonella, Klebsiella, Pseudomonas, Vibrio, Helicobacter, Haemophilus, Halomonas, Bacterioides, Prevotella, Bartonella, Porphyromonas, Actinomyces, Streptomyces, Corynebacterium, Propionibacterium, Mycobacterium, Caulobacter, Bradyrhizobium, Agrobacterium, Rhodobacter, Rhodopseudomonas, Magnetospirillum, Magnetobacterium, Acetobacter, Zymomonas, Rikettsia, Eleftheria, Saccharonmyces, Schizosacchoromyces, Scheffereromyces, Zygosaccharomyces, Yarrowia, Pichia, Dekkera, Kluyveromyces, Candida, Metschnikowia, and Torulaspora.

5. A method according to claim 1, wherein said modified microbe is derived from a parent microorganism that comprises properties that are beneficial for improved survivability in the lung, selected from species of Bifidobacterium, Gardgnerella, Clostridium, Deinococcus, Faecalibacterium, Anaerobacter, Coprobacillus, Oxobacter, Sporobacter, Eubacterium, Heliobacterium, Oscillospira, Peptococcus, Dehalobacter, Butyrivibrio, Coprococcus, Lachnospira, Ruminococcus, Bacteroides, Prevotella, Bartonella, Bdellovibrio, Micabibrio, Vampirobibrio, Vampirococcus, Daptobacter, Lysobacter, Myxococcus, Aristabacter, Cytophaga, Bacillus, Paenibacillus, Staphylococcus, Enterococcus, Enterobacter, and Escherichia.

6. A method according to claim 1, wherein said lung condition comprises a microbial infection or a eukaryotic infection.

7.-14. (canceled)

15. A method according to claim 1, wherein said lung condition comprises a viral infection.

16. (canceled)

17. A method according to claim 1, wherein the lung condition comprises fibrotic disease (FD), cystic fibrosis (CF), idiopathic pulmonary fibrosis (IPF), or interstitial pneumonia.

18. (canceled)

19. A method according to claim 1, wherein said lung condition comprises an inflammatory disease, an autoimmune disease, or cancer.

20.-23. (canceled)

24. A method according to claim 1, wherein said modified microbe is administered in combination with at least one other treatment to treat, prevent, or ameliorate at least one symptom of said lung condition.

25. A method according to claim 24, wherein said at least one other treatment comprises an antibiotic, a bacteriophage, an antibody, a peptide, an enzyme, a sugar, a glycopolymer, a bacteriocin, or spores.

26. A method according to claim 1, wherein said therapeutically or prophylactically effective amount comprises one or more modified microbe formulated in a dry powder or as a solution.

27. A method according to claim 26, wherein the dry powder or solution is administered by inhalation or is formulated for regional deposition in the lung.

28. (canceled)

29. A method according to claim 27, wherein the contents of the dry powder or solution are released in the large airways, small airways, or bronchioles.

30. A method according to claim 1, wherein said therapeutically or prophylactically effective amount comprises one or more modified microbe administered intranasally.

31. A pharmaceutical composition, comprising a therapeutically or prophylactically effective amount of a modified microbe, and a pharmaceutically acceptable carrier, formulated for treatment or prevention of a lung condition.

32. A pharmaceutical composition according to claim 31, formulated for regional deposition in the lung or for release in the sinus cavity.

33. (canceled)

34. A dosage form comprising a therapeutically or prophylactically effective dose of a pharmaceutical composition according to claim 31.

35. A dosage form according to claim 34, in the form of a dry powder or solution.

36. A kit comprising a dosage form according to claim 35, and instructions for use in a method of treatment or prevention of a lung condition.
